(19) United States
(12) Patent Application Publication
(10) Pub. No.: US 2018/0027834 A1
(43) Pub. Date: Feb. 1, 2018

(54) MICROCAPSULES COMPRISING LUTEIN OR LUTEIN ESTER

(71) Applicant: BASF SE, Ludwigshafen (DE)

(72) Inventors: Nina MUSAEUS, Hellerup (DK); Inge-Lise KRYLBO, Søborg (DK)

(21) Appl. No.: 15/548,964

(22) PCT Filed: Feb. 8, 2016
(86) PCT No.: PCT/EP2016/052619

§ 371 Date: Aug. 4, 2017

(30) Foreign Application Priority Data
Feb. 6, 2015 (EP) 15154154.7

(51) Int. Cl. A23C 9/152; A23L 29/25; A23P 10/30; C09B 61/00; A61K 9/20; A61K 9/50; C09B 67/02; A23L 33/105; A61K 31/047

(52) U.S. Cl. CPC A23C 9/152; A23L 33/105; A23L 29/25; A23P 10/30; A61K 31/047; A61K 9/2081; A61K 9/5036; A61K 9/5089; C09B 67/0097; C09B 61/00; A23V 2002/00

(57) ABSTRACT

The invention relates to microcapsules comprising at least one active substance selected from lutein and lutein esters embedded in a matrix comprising gum acacia and optionally one or more other matrix components, wherein the content of said at least one active substance calculated as free lutein is from 0.5 to 25% of total weight of the microcapsule, and which microcapsule does not comprise any added emulsifier.

The invention further relates to a process of preparing the microcapsule as well as uses and products comprising the microcapsule.

### FIELD OF THE INVENTION

The present invention relates to a microcapsule comprising lutein or lutein esters as active substance embedded in a hydrocolloid matrix of native gum acacia, a process for preparing such microcapsules as well as applications thereof and products comprising such microcapsules.

### BACKGROUND OF THE INVENTION

Lutein is a xanthophyll and a naturally occurring carotenoid found in plants, such as flowers, in particular Marigold flowers, and green leafy vegetables. Lutein may for instance be extracted from the petals of marigold, spinach, kale and broccoli. Marigold is in particular rich in lutein, and it is found as lutein esters with fatty acids. Lutein can be used as yellow pigment in all kind of compositions, such as functional foods and health care products, and it has well known pharmacological effects and applications.

Lutein as free lutein has the chemical structure

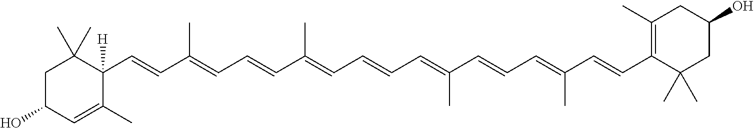 [SMILES: CC1=C[C@H](O)CC(C)(C)[C@H]1/C=C/C(C)=C/C=C/C(C)=C/C=C/C=C(C)/C=C/C=C(C)/C=C/C1=C(C)C[C@@H](O)CC1(C)C]

The typical lutein esters found in Marigold are the mono- or dipalmitate, and these esters have properties different from the free lutein. The molecular weight of free lutein is about 569 g/mol, whereas the molecular weight of the dipalmitate is about 1046 g/mol.

The melting point of free lutein is about 190° C., whereas the melting range of the mixed naturally occurring lutein esters is from about 50 to about 80° C.

Chinese patent application 102389108 A discloses lutein ester microcapsule powder comprising lutein ester crystals, antioxidants and emulsifying agents for both the oil-phase and the aqueous-phase, filler, wall material and oil, and a method for preparing the same.

The filler material applied is modified gum arabic or modified starch, such as OSA modified gum Arabica or OSA modified starch. The application claims that both a water soluble and a oil soluble emulsifier are needed to produce the microcapsule powder. Using emulsifiers in the formulation has the disadvantage that air will be trapped into the powder particles and form hollow spheres. The included air and the porosity of the microcapsules leads to chemical degradation of the lutein or lutein ester. Using emulsifiers furthermore has the disadvantage that the preparation is more expensive and time consuming to produce because the emulsifiers have to be mixed thoroughly with the other ingredients in the aqueous and the oil phase, respectively. Furthermore the list of ingredients in the powder formulation increases.

EP 1 794 238 81 discloses a carotenoid-containing dry powder comprising one or more carotenoids such as crystalline lutein obtainable by a microencapsulation process using isomalt and a protective colloid such as modified starch as encapsulation material, wherein the initial suspension of crystalline carotenoids is grounded.

EP 1 898 721 B1 discloses an aqueous carotenoid-containing suspension comprising at least one or more carotenoids such as crystalline lutein, modified starch and sucrose, wherein the initial suspension of crystalline carotenoids is grounded.

WO2014/154788 discloses a powderous composition comprising particles of lutein and maltodextrin as matrix material.

The main object of the present invention is to provide improved products based on lutein or lutein ester concentrates and gum acacia which shall have a cleaner label and fewer ingredients. They shall have or create in the final product a more intense and longer lasting colour impression and they shall be more stable and less sensitive to oxidation compared to state of the art products. They shall exhibit a greater chemical stability and be more natural in composition. They shall be produced in an easier and more cost effective way.

Another object is to provide improved products based on lutein or lutein ester concentrates and gum acacia which can be used in tablets, dietary supplements and dairies, such as milk products with high stability due to proper encapsulation of the lutein and/or lutein ester and with improved oxygen barrier properties.

It is also an object to provide products with good colouring properties.

Yet another object is to provide a cost effective method for producing such products.

Finally, it is an object to provide products free of mammalian or fish protein.

### SUMMARY OF THE INVENTION

The present invention relates to a microcapsule comprising at least one active substance selected from lutein and lutein esters, embedded in a matrix comprising native gum acacia and optionally one or more other matrix components, wherein the content of said at least one active substance calculated as free lutein is from 0.5 to 25% of total weight of the microcapsule, and which microcapsule does not comprise any added emulsifier.

In particular, the microcapsule does not comprise any added oil phase emulsifier.

It has surprisingly been found that the microcapsule of the invention can be provided without any added dispersing agent in addition to the gum acacia. Accordingly, the gum acacia is the sole agent with dispersing properties present in the microcapsule. Traditionally, and according to prior art, also a classical emulsifier is included in a microcapsule to ensure a sufficiently small particle size, and a small particle size is important for the appearance and colour of the final product comprising the microcapsules, such as dairies and food. The absence of any added classical emulsifier has also the advantage in comparison with prior art microcapsules comprising lutein and lutein esters that foaming during preparation is avoided. Foaming during preparation will lead to inclusion of air in the microcapsules which will decrease the chemical stability of the lutein or lutein ester in the final product. Finally, a product without any added emulsifier provides the advantage of a cleaner label.

The present invention in another aspect relates to a process of preparing a microcapsule according to the invention, which process comprises the steps of
melting or dissolving lutein or lutein ester concentrate(s),
providing an aqueous solution of native gum acacia and said optionally other matrix components,
mixing said aqueous solution and said melted or dissolved lutein or lutein ester concentrate(s),
homogenising the resulting preparation without addition of an emulsifier,
finely dividing and drying the mixture thus obtained to prepare a mass of particles each containing lutein or lutein ester(s) embedded in native gum acacia.

In a third aspect the invention relates to microcapsules according to the invention obtainable according to the process of the invention.

In a fourth aspect the invention relates to products comprising the microcapsule of the invention, in particular tablets, dietary supplements and food.

### Definitions

In the context of the current invention, the following terms are meant to comprise the following, unless defined elsewhere in the description.

Lutein ester concentrate is a dark orange-brown oleoresin or a granular powder having a melting range of approximately 50-80° C., It typically comprises 70-85% lutein ester, corresponding to about half the amount of free lutein. It can be dissolved or melted in oil.

Lutein ester concentrate complies with the EFSA (European Food Safety Authority) specification for lutein (Directive 2008/128/EC (E 161b). The main colouring principle of lutein consists of carotenoids of which lutein and its fatty esters account for the major part, Variable amounts of other carotenes and xanthophyll esters, such as zeaxanthin ester, are also present in the concentrate. Lutein may contain fats, oils and waxes naturally occurring in the plant material. Lutein ester concentrate contains min. 60% total carotenoid esters.

Native gum acacia is in this context defined as a protective hydrocolloid with dispersing properties. The gum acacia is native in the sense that it is chemically unmodified. In the context of the present invention it is not to be understood as a classical emulsifier (surfactant) in accordance with the definition below. Gum Acacia is a dried exudate from the stem and branches of acacia Senegal or acacia Seyal. Gum acacia is commercially available in different qualities, Qualities based on acacia Senegal are preferred for encapsulation.

An emulsifier is defined as a substance with a hydrophilic head and a hydrophobic tail. Emulsifiers can be divided into non-ionic, anionic and cationic emulsifiers. Depending on the HLB-value (hydrophilic-lipophilic balance) emulsifiers can be either oil soluble (low HLB values) or water soluble (high HLB values), Adding both types to an emulsion often works synergistic. Typical emulsifiers allowed in food products comprise glycerol fatty acid esters, sucrose fatty acid esters, sorbitan fatty acid esters, lecithins, ascorbyl palmitate and ascorbyl stearate.

Dispersing means mixing one phase (the continuous phase) with a second phase (the disperse phase) where the two phases not being miscible to prepare a dispersion. The nature of each phase can be liquid, solid or gaseous.

Homogenising means treating a dispersion in order to reduce the size of the droplets/particles of the disperse phase.

### DETAILED DESCRIPTION OF THE INVENTION

In one embodiment of the microcapsule of the invention the content of said at least one active substance, calculated as free lutein, is from 1 to 20% of total weight of the microcapsule, preferably from 3 to 15%, more preferably from 4 to 13%, for instance from 5 to 10% of total weight of the microcapsule.

In a second embodiment of the microcapsule of the invention the native gum acacia is a quality gained from Acacia Senegal.

In a third embodiment of the microcapsule of the invention it comprises at least one antioxidant e.g. selected from the group essentially consisting of or comprising t-butylhydroxytoluene (BHT), t-butylhydroxyanisole (BHA), ascorbic acid, sodium ascorbate, citric acid, sodium citrate, EDTA or its salts, tocopherols, TBHQ, ethoxyquine, propyl gallate, and extracts from herbs, i.a. rosemary or oregano extract.

In a forth embodiment of the microcapsule it comprises at least one plasticizer, e.g. selected from the group essentially consisting of or comprising carbohydrates and carbohydrate alcohols, examples of which are sucrose, glucose, fructose, lactose, invert sugar, glucose syrup, sorbitol, mannitol, trehalose, tagatose, pullulan, Raftilose (oligofructose), dextrin, maltodextrin, glycerin, and mixtures thereof.

In a fifth embodiment the microcapsule comprises lutein ester(s) as active substance. In comparison with prior art products comprising microcapsules of free lutein this microcapsule has the further advantage that the lutein ester(s) is the naturally occurring form of the lutein. Producing free lutein from a marigold extract requires saponification under harsh conditions. This processing step is avoided when using lutein ester(s) in the microcapsule. In a particular form of this embodiment the microcapsule has been prepared from non-crystalline lutein ester concentrate(s).

In a sixth embodiment the microcapsule is prepared from an emulsion of melted or dissolved lutein or lutein ester concentrate(s) in an aqueous solution of native gum acacia in the absence of an emulsifier, whereby said lutein or lutein ester concentrate(s) is optionally melted or dissolved in an edible oil, such as a vegetable oil.

In a seventh embodiment the microcapsule is prepared from non-crystalline lutein ester.

In a eighths embodiment of the microcapsule of the invention the lutein/lutein ester droplets have an average size D[4;3] determined by Fraunhofer diffraction of from 0.02 to 100 μm, preferably 0.05 to 50 μm, more preferred 0.1 to 5 μm or 0.2 to 1.5 μm; and in particular from 0.1 to 1 μm. The term D[4;3] is explained in the introduction to the examples.

The microcapsule may further contain conventional additives e.g. selected from the group essentially consisting of or comprising as anti-caking agents, e.g. tri-calcium phosphate and silicates, i.a. silicon dioxide and sodium aluminium silicate.

The dividing and drying of the mixture of the oil-in-water preparation to produce a mass of particles can be done in any conventional way, such as spray cooling, modified spray cooling, spray drying, modified spray drying or sheet drying and crushing, see e.g. WO 91/06292 A1.

In one embodiment of the process of the invention the lutein or lutein ester concentrate(s) is melted or dissolved in vegetable oil, e.g. selected from the group essentially consisting of or comprising sunflower oil, olive oil, cotton seed oil, safflower oil, MCT oil, palm oil or hydrogenated palm oil. Melting or dissolving the lutein or lutein ester in an oil facilitates dispersing and homogenising and reduces the temperature to be applied.

The process of the invention may in a second embodiment comprise a further step of homogenisation, such as high pressure homogenisation.

In a third embodiment of the process of the invention the aqueous solution of gum acacia is added to the melted or dissolved lutein or lutein ester concentrate(s) before homogenisation. Adding the aqueous phase to the oil phase minimizes physical loss of the lutein or lutein ester.

In a forth embodiment of the process the melted or dissolved lutein or lutein ester concentrate(s) is added to the aqueous solution of gum acacia before homogenisation.

In a fifth embodiment of the process the lutein or lutein ester concentrate(s) is added to the aqueous solution of gum acacia and melted during heating before homogenisation. This is preferred if the lutein or lutein ester is not melted or dissolved in oil before homogenisation because it is a more simple process and the physical loss is minimized.

In a sixth embodiment of the process of the invention the homogenisation continues until the lutein/lutein ester droplets have an average size D[4;3] determined by Fraunhofer diffraction of from 0.02 to 100 μm, preferably 0.05 to 50 μm, more preferred 0.1 to 5 μm or 0.2 to 2 μm; and in particular from 0.1 to 1 μm. The term D[4;3] is explained in the introduction to the examples.

In a seventh embodiment of the process lutein ester concentrate(s) is melted or dissolved. Lutein ester has a lower melting point than free lutein, and this makes it possible, in this embodiment of the invention, to melt or dissolve the lutein ester under atmospheric pressure and to use it directly in the process. This process includes less harsh conditions, and is therefore more cost effective than the process which uses free lutein.

Melting the lutein or lutein ester concentrate(s) is cost-effective since use of solvent can be economized. This in particular holds for lutein esters having a lower melting point compared to free lutein and thus need only small heating energy for melting.

The present invention also relates to a product comprising microcapsules of the invention or microcapsules produced according to the invention. Examples of such products are a tablet, a dietary supplement, a dairy, a food, a food supplement, a pharmaceutical or veterinary product, a feed or feed supplement, a beverage, a personal care product or a household product.

### EXAMPLES

Determination of Content of Lutein Ester and Free Lutein

The content of lutein ester and free lutein in the microcapsules is determined as follows: The lutein or lutein ester is released from the microcapsules under mild alkaline conditions using alkalase and heat. The lutein or lutein ester ester is extracted by means of ethanol and diethyl ether in a ratio of 2:5 and an aliquot of this extraction is dissolved in a known volume of ethanol. The UV/Vis absorbance is measured at a specific wavelength and the concentration is calculated from a known extinction coefficient via Lambert-Beers equation. When using the absorbance in lambda (max)=approx. 446 nm, the content of lutein ester in microcapsules containing lutein ester can be calculated by using the extinction coefficient E^{1%}_{1 cm}=1373. The corresponding content of free lutein can be calculated form the same measurement by using the extinction coefficient E^{1%}_{1 cm}=2550. For microcapsules containing free lutein the extinction coefficient E^{1%}_{1 cm}=2550 is used to calculate the content of free lutein.

Measuring of Particle Size (Oil Droplet Size)

Homogenisation is performed in conventional homogenisation equipment. Homogenisation takes place until the oil droplets have the intended average size D[4;3] determined by Fraunhofer diffraction. The term D[4;3] refers to the volume-weighted average diameter (see Operators Guide, Malvern Mastersizer 2000, Malvern Instruments Ltd., 1998/1999, UK, Chapter 6, page 6.3).

Measuring of Tablet Stability.

Multivitamin mineral tablets with a content of approximately 2 mg lutein ester (or 1 mg lutein) per tablet were prepared. The tablets were filled in HOPE containers sealed with an alumina lid. The tablets were stored at 40° C. and 75% relative humidity for 6 months. The lutein ester content was analyzed in each case after storage for 3 and 6 months.

### Example 1

514 g native gum acacia, 171 g sucrose and 18 g sodium ascorbate were dissolved in 600 g water at 65° C. during stirring. 180 g lutein ester concentrate and 17.2 g mixed tocopherols (70% concentrate) were added during stirring followed by homogenisation until the lutein ester droplets had an average particle size D[4;3] of less than 1.0 μm. The viscosity was adjusted with water and the dispersion was sprayed into native corn starch containing silicon dioxide as a flow agent. The formed particles were dried in air at 40-150° C. until the water content in the powder was below 5%.

The resulting dried powder had a content of 11.3% lutein esters corresponding to 6.08% free lutein determined by UV/Vis spectroscopy.

### Example 2

In vessel A 1541 g native gum acacia, 382 g sucrose and 54 g sodium ascorbate were dissolved in 1800 g water at 65° C. during stirring. In vessel B 540 g lutein ester concentrate was melted together with 137 g MCT oil and 48.2 g mixed tocopherols (70% concentrate) at 60-90° C. The oil phase from vessel B was added to the aqueous phase in vessel A during stirring followed by homogenisation until the lutein ester droplets had an average particle size D[4;3] of less than 1.0 μm. The viscosity was adjusted with water and the dispersion was sprayed into native corn starch, containing silicon dioxide as a flow agent. The formed particles were dried in air at 40-150° C. until the water content in the powder was below 5%.

The resulting dried powder had a content of 10.9% lutein esters corresponding to 5.87% free lutein determined by UV/Vis spectroscopy.

### Example 3

In vessel A 1541 g native gum acacia, 382 g sucrose and 54 g sodium ascorbate were dissolved in 1800 g water at 65° C. during stirring. In vessel B 540 g lutein ester concentrate was melted together with 137 g hydrogenated palm oil and 48.2 g mixed tocopherols (70% concentrate) at 60-90° C. The oil phase from vessel B was added to the aqueous phase in vessel A during stirring followed by homogenisation until the lutein ester droplets had an average particle size D[4;3] of less than 1.0 μm. The viscosity was adjusted with water and the dispersion was sprayed into native corn starch containing silicon dioxide as a flow agent. The formed particles were dried in air at 40-150° C. until the water content in the powder was below 5%.

The resulting dried powder had a content of 12.2% lutein esters corresponding to 6.57% free lutein determined by UV/Vis spectroscopy.

### Example 4

In vessel A 1541 g native gum acacia, 382 g sucrose and 54 g sodium ascorbate were dissolved in 1800 g water at 65° C. during stirring. In vessel B 540 g lutein ester concentrate was melted together with 137 g hydrogenated palm oil and 48.2 g mixed tocopherols (70% concentrate) at 60-90° C. The oil phase from vessel B was added to the aqueous phase in vessel A during stirring followed by homogenisation until the lutein ester droplets had an average particle size D[4;3] of less than 1.0 μm. The viscosity was adjusted with water and the dispersion was passed through a high pressure homogenizer at 700 bar and sprayed into native corn starch containing silicon dioxide as a flow agent. The formed particles were dried in air at 40-150° C. until the water content in the powder was below 5%.

The resulting dried powder had a content of 10.0% lutein esters corresponding to 5.38% free lutein determined by UV/Vis spectroscopy.

### Example 5

In vessel A 1750 g native gum acacia, 584 g sucrose and 62.5 g sodium ascorbate were dissolved in 2700 g water at 65° C. during stirring. In vessel B 625 g lutein ester concentrate was melted together with 62.5 g sunflower oil and 44.6 g mixed tocopherols (70% concentrate) at 60-90° C. The aqueous phase from vessel A was added to the oil phase in vessel B during stirring followed by homogenisation until the lutein ester droplets had an average particle size D[4;3] of less than 1.0 μm. The viscosity was adjusted with water and the dispersion was passed through a high pressure homogenizer at 800 bar and sprayed into native corn starch containing tricalcium phosphate as a flow agent. The formed particles were dried in air at 40-150° C. until the water content in the powder was below 5%.

The resulting dried powder had a content of 8.6% lutein esters corresponding to 4.63% free lutein determined by UV/Vis spectroscopy.

### Example 6

1743 g native gum acacia, 581 g sucrose and 50 g sodium ascorbate were dissolved in 2000 g water at 65° C. during stirring. 500 g lutein ester concentrate, 50 g sunflower oil and 35.7 g mixed tocopherols (70% concentrate) were added during stirring followed by homogenisation until the lutein ester droplets had an average particle size D[4;3] of less than 1.0 μm. The viscosity was adjusted with water and the dispersion was passed through a high pressure homogenizer at 800 bar and sprayed into native corn starch containing tri-calcium phosphate as a flow agent. The formed particles were dried in air at 40-150° C. until the water content in the powder was below 5%.

The resulting dried powder had a content of 10.1% lutein esters corresponding to 5.44% free lutein determined by UV/Vis spectroscopy.

### Example 7

1743 g native gum acacia, 581 g sucrose and 50 g sodium ascorbate were dissolved in 1700 g water at 65° C. during stirring. 500 g lutein ester concentrate, 50 g sunflower oil and 35.7 g mixed tocopherols (70% concentrate) were added during stirring followed by homogenisation until the lutein ester droplets had an average particle size D[4;3] of less than 1.0 The viscosity was adjusted with water and the dispersion was passed through a high pressure homogenizer at 800 bar and sprayed into native corn starch containing tri-calcium phosphate as a flow agent. The formed particles were dried in air at 40-150° C. until the water content in the powder was below 5%.

The resulting dried powder had a content of 9.3% lutein esters corresponding to 5.0% free lutein determined by UV/Vis spectroscopy.

### Example 8

In vessel A 1750 g native gum acacia, 584 g sucrose and 62.5 g sodium ascorbate were dissolved in 1800 g water at 65° C. during stirring. In vessel B 625 g lutein ester concentrate was melted together with 62.5 g sunflower oil and 44.6 g mixed tocopherols (70% concentrate) at 6090° C. The aqueous phase from vessel A was added to the oil phase in vessel B during stirring followed by homogenisation until the lutein ester droplets had an average particle size D[4;3] of less than 1.0 μm. The viscosity was adjusted with water and the dispersion was passed through a high pressure homogenizer at 800 bar and sprayed into native corn starch containing tri-calcium phosphate as a flow agent. The formed particles were dried in air at 50-150° C. until the water content in the powder was below 5%.

The resulting dried powder had a content of 10.6% lutein esters corresponding to 5.70% free lutein determined by UV/Vis spectroscopy.

The microcapsules prepared according to the examples were tested for stability in tablets and milk.

### Example 9

Tablet Preparation

The chemical stability of the lutein ester microcapsule powder was tested by means of multivitamin mineral tablets having a content of about 2 mg of lutein ester per tablet. The tablets were packaged in HDPE containers whose lid was sealed with heat-sealed aluminum foil. The tablets were store d at 40° C. and 75% relative humidity for 6 months. The lutein ester content was analyzed in each case after storage for 3 and 6 months. The results were as shown in Table 1:

Tablet stability data, storage at 40° C./75% RH, available data

| | T = 0 % | 3 months % | 6 months % |
|---|---|---|---|
| Ex | remaining | remaining | remaining |
| 1 | 100 | 97 | 98 |
| 2 | 100 | 95 | 93 |
| 3 | 100 | 94 | 92 |
| 4 | 100 | 93 | 89 |
| 6 | 100 | 99 | 103 |
| 8 | 100 | 100 | 103 |

### Example 10

Milk Preparation

An amount of lutein ester microcapsule powder corresponding to a concentration of 20 ppm lutein was dissolved in cold milk. The solution was heated to 60° C. and homogenized followed by pasteurization at 95° C. in 10 minutes. The milk was filled into bottles and stored cold (below 10° C.) for 3 weeks. The colloidal stability (ring formation) was evaluated visually every week.

The powders comprising microcapsules prepared according to example 4 were tested in milk. After 2 weeks storage no ring formation in milk was observed i.e. the sample had sufficient stability in this application.

The invention is not reduced to the previously given examples but can be varied in many fold ways. For instance instead of lutein or lutein esters or in combination with said lutein or lutein esters the following carotenoids or its esters can also be used: zeaxanthin, beta-carotene, alpha-carotene, lycopene, astaxanthin, canthaxanthin, beta-cryptoxanthin, citranaxanthin and beta-apo-8′-carotenoids.

1.-18. (canceled)

19. A microcapsule comprising at least one active substance selected from lutein and lutein esters embedded in a matrix comprising native gum acacia and optionally one or more other matrix components, wherein the content of said at least one active substance calculated as free lutein is from 0.5 to 25% of total weight of the microcapsule, and which microcapsule does not comprise any added emulsifier.

20. The microcapsule according to claim 19, wherein the content of said at least one active substance calculated as free lutein is from 1 to 20% of total weight of the microcapsule.

21. The microcapsule according to claim 19, wherein the content of said at least one active substance calculated as free lutein is from 3 to 15% of total weight of the microcapsule.

22. The microcapsule according to claim 19, wherein the content of said at least one active substance calculated as free lutein is from 4 to 13% of total weight of the microcapsule.

23. The microcapsule according to claim 19, wherein the content of said at least one active substance calculated as free lutein is from 5 to 10% of total weight of the microcapsule.

24. The microcapsule according to claim 19 further comprising at least one antioxidant and/or plasticizer.

25. The microcapsule according to claim 19, wherein said gum acacia is a quality gained from Acacia Senegal.

26. The microcapsule according to claim 19 prepared from an emulsion of melted or dissolved lutein or lutein ester concentrate(s) in an aqueous solution of said native gum acacia in the absence of an emulsifier, wherein said lutein or lutein ester concentrate(s) is optionally melted or dissolved in an edible oil.

27. The microcapsule according to claim 19 prepared from non-crystalline lutein ester.

28. A process of preparing the microcapsule according to claim 19, which process comprises the steps of
melting or dissolving lutein or lutein ester concentrate(s),
providing an aqueous solution of native gum acacia and said optionally other matrix components,
mixing said aqueous solution and said melted or dissolved lutein or lutein ester concentrate(s),
homogenizing the resulting preparation without addition of an emulsifier,
finely dividing and drying the mixture thus obtained to prepare a mass of particles each containing lutein or lutein ester(s) embedded in native gum acacia.

29. The process according to claim 28 wherein the lutein ester concentrate(s) is melted or dissolved in edible oil.

30. The process according to claim 28 wherein the lutein ester concentrate(s) is melted or dissolved in sunflower oil, olive oil, cotton seed oil, safflower oil, MCT oil, palm oil or hydrogenated palm oil.

31. The process according to claim 28 wherein said aqueous solution of native gum acacia is added to said melted or dissolved lutein or lutein ester concentrate(s) before homogenization.

32. The process according to claim 28 wherein said melted or dissolved lutein or lutein ester concentrate(s) is added to said aqueous solution of native gum acacia before homogenization.

33. The process according to claim 28 comprising a further step of homogenization.

34. The process according to claim 28 comprising a further step of high pressure homogenization.

35. A microcapsule comprising at least one active substance selected from lutein and lutein esters embedded in a matrix comprising native gum acacia and optionally one or more other matrix components, wherein the content of said at least one active substance calculated as free lutein is from 0.5 to 25% of total weight of the microcapsule, and which microcapsule does not comprise any added emulsifier obtainable by a process comprising the steps of
melting or dissolving lutein or lutein ester concentrate(s),
providing an aqueous solution of said native gum acacia and said optionally other matrix components,
mixing said aqueous solution and said melted or dissolved lutein or lutein ester concentrate(s),
homogenizing the resulting preparation without addition of an emulsifier,
finely dividing and drying the mixture thus obtained to prepare a mass of particles each containing lutein or lutein ester(s) embedded in native gum acacia.

36. A product comprising microcapsules according to claim 19.

37. The product according to claim 36, wherein the product is a tablet, a dietary supplement, a food, a food supplement, a dairy, a pharmaceutical or veterinary product, a feed or feed supplement, a beverage, a personal care products or a household product.

38. A tablet, dietary supplement or food comprising the microcapsules according to claim 19 wherein said native gum acacia is a quality gained from Acacia Senegal.
